(19) United States
(12) Patent Application Publication
(10) Pub. No.: US 2024/0198042 A1
(43) Pub. Date: Jun. 20, 2024

(54) Acute Central Venous Catheter Symmetric Filler

(71) Applicant: Bard Access Systems, Inc., Salt Lake City, UT (US)

(72) Inventor: Juan Sepulveda, Centerville, UT (US)

(21) Appl. No.: 18/081,480

(22) Filed: Dec. 14, 2022

(51) Int. Cl. A61M 25/00

(52) U.S. Cl. CPC A61M 25/0043; A61M 25/0026

(57) ABSTRACT

A system includes a catheter and one or more fillers, or plugs, configured to block a lumen of the catheter. The catheter may include an elongate tube having a distal end and defining a plurality of lumens. A distal tip structure may be located adjacent to the distal end. A plug may be configured to be positioned within a first portion of a first lumen of the plurality of lumens proximal the distal tip. The plug may include an oval cross-sectional shape and be formed of a biocompatible polymeric material such as silicone, nylon, polyurethane, polyethylene terephthalate, a latex, a plastic, a thermoset, or a thermoplastic elastomer. The oval cross-sectional shape may have first and a second vertices, where the first plug is configured such that either of the vertices may be positioned near an upper side of the first lumen without causing bulging of a wall of the lumen.

### BACKGROUND

During manufacture of a catheter, a process of catheter tipping, or end forming, is undertaken to shape the end of a thermoplastic tube, which may be performed with the assistance of a thermally conductive mold. Prior to tipping a catheter, the distal portions of one or more lumens (e.g., the proximal and medial lumens) must be plugged to prevent the creation of bacteria or thrombogenicity in the space between lumen exit and the tip of the catheter. This is typically done by way of a monofilament that is inserted into these lumens. With some catheters, the proximal and medial lumens of the catheter shaft have a unique kidney-like cross-sectional shape, the filler geometry must attempt to fill most, if not, all of the lumen area.

Current implementations of monofilaments include monofilaments having a geometry that attempts to replicate the shape of the lumen. For instance, current monofilaments have a substantially kidney-like cross-sectional shape. While this geometry would in theory effectively fill such lumens, numerous disadvantages of this geometry have been observed in practice. First, the complexity of the geometry of the monofilament prevents the extrusion vendor of the monofilament from exactly replicating the geometry of the kidney-like cross-sectional shaped lumen, which affects the effectiveness of the ability of the plug to fill the lumen. Second, as the kidney-shaped cross sectional shape is not symmetric along any axis, during manufacturing the operators often load the monofilament into the lumen in the wrong direction. This causes a bulge or numerous bulges in the catheter shaft or internal lumen walls.

Thus, what is needed is a plug having an improved geometry that does not suffer from the disadvantages of the current fillers by simplifying the shape of the extrusion die, which allows for improved extrusion accuracy of the monofilament vendor and avoidance of misalignment of the plug within the lumen during manufacture.

### SUMMARY

Briefly summarized, embodiments of the present invention are directed a system, comprising a catheter and a first plug. The catheter includes an elongate tube including a distal end and defining a plurality of lumens, and a distal tip structure located adjacent the distal end. The first plug is configured for positioning in a first portion of a first lumen of the plurality of lumens proximal the distal tip, wherein the first plug is defined as having an oval cross-sectional shape.

In some embodiments, a second plug positioned in a second portion of one of the plurality of lumens, wherein the second plug is defined as having the oval cross-sectional shape, In some instances, the first plug and second plug are positioned different lumens. In some examples, the first plug is formed of a biocompatible polymeric material such as one or more of silicone, nylon, polyurethane, polyethylene terephthalate, latex, plastic, thermoset, or thermoplastic elastomer. In some instances, the first plug is formed of a radiopaque material.

The first plug includes a cross-section having a first line of symmetry, where a length of the first line of symmetry is within a range of 0.035-0.050 inches. In some particular embodiments, the length of the first line of symmetry is 0.041±0.002 inches. Additionally, the first plug includes a cross-section having a second line of symmetry, where a length of the second line of symmetry is within a range of 0.0175-0.035 inches. In some particular examples, the length of the second line of symmetry is 0.0250±0.0015 inches.

The oval cross-sectional shape of the first plug includes a first vertex and a second vertex, wherein the first plug is configured such that either the first vertex or the second vertex may be positioned near an upper side of the first lumen without causing bulging of a wall of the lumen.

In some embodiments, the first portion is located of the first lumen in a distal section of the catheter, the first portion is located of the first lumen in a medial section of the catheter, or the first portion is located of the first lumen in the proximal section of the catheter. In some embodiments, the plurality of lumens includes the first lumen and a second lumen arranged in a generally double-D configuration, the corners of each lumen being rounded. In some examples, the plurality of lumens includes a first lumen, a second lumen, and a third lumen, wherein each lumen is separated by at least one septum from another lumen.

In some examples, the plurality of lumens includes a first lumen, a second lumen, a third lumen, and a fourth lumen, wherein at least two of the plurality of lumens are bisected by one of a minor axis and a major axis of the catheter, at least one of the plurality of lumens being power injectable. In some instances, a proximal portion of the elongate tube defines a circular cross-section and a distal portion of the elongate tube defines an elliptical cross-section. In some examples, a proximal portion of the elongate tube defines an elliptical cross-section and a distal portion of the elongate tube defines a circular cross-section. In some instances, the plurality of lumens include a cross-sectional profile that is generally triangular or kidney shaped.

These and other features of embodiments of the present invention will become more fully apparent from the following description and appended claims or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A and 1B are perspective and cross-sectional views, respectively, of a catheter assembly configured in accordance with one embodiment;

FIG. 2 illustrates a cross-sectional view of a filler in accordance with an embodiment;

FIG. 3 is a cross-sectional view of a catheter tube including the filler of FIG. 2 in accordance with an embodiment;

FIG. 4 is a cross-sectional view of a filler in accordance with an embodiment;

FIG. 5 is a cross-sectional view of a catheter tube including the filler of FIG. 4 in accordance with an embodiment;

FIG. 6 is a cross-sectional view of a catheter tube including a filler in accordance with one embodiment;

FIG. 7 is a cross-sectional view of a catheter tube including a plurality of fillers in accordance with one embodiment;

FIG. 8 is a cross-sectional view of a catheter tube including a plurality of fillers having different cross-sectional shapes in accordance with one embodiment;

FIG. 9 is a cross-sectional view of a catheter tube including a plurality of fillers having the same cross-sectional shape in various sizes in accordance with one embodiment;

FIG. 10 is a cross-sectional view of a catheter tube including a plurality of fillers in accordance with one embodiment

FIGS. 11 and 12 are perspective and cross-sectional views, respectively, showing insertion and disposal of the catheter tube of FIG. 10 within an introducer, according to one embodiment;

FIGS. 13A and 13B are side and top views, respectively, of a catheter assembly in accordance with one embodiment;

FIGS. 14A and 14B are cross-sectional views of the catheter assembly of FIG. 13B including fillers; and

FIG. 15 illustrates an exemplary method of coupling a distal tip structure with a catheter body, in accordance with one implementation.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a widget disclosed herein includes a portion of the widget intended to be near a user (e.g., a holder of the widget). Likewise, a “proximal length” of, for example, the widget includes a length of the widget intended to be near the user. A “proximal end” of, for example, the widget includes an end of the widget intended to be near the user. The proximal portion, the proximal end portion, or the proximal length of the widget can include the proximal end of the widget; however, the proximal portion, the proximal end portion, or the proximal length of the widget need not include the proximal end of the widget. That is, unless context suggests otherwise, the proximal portion, the proximal end portion, or the proximal length of the widget is not a terminal portion or terminal length of the widget.

With respect to “distal,” a “distal portion” or a “distal end portion” of, for example, a widget disclosed herein includes a portion of the widget intended to be opposite the user with respect to the proximal portion (e.g., “away” from the user). Likewise, a “distal length” of, for example, the widget includes a length of the widget intended to be opposite the proximal portion and away from the user. A “distal end” of, for example, the widget includes an end of the widget intended to be opposite the proximal end. The distal portion, the distal end portion, or the distal length of the widget can include the distal end of the widget; however, the distal portion, the distal end portion, or the distal length of the widget need not include the distal end of the widget. That is, unless context suggests otherwise, the distal portion, the distal end portion, or the distal length of the widget is not a terminal portion or terminal length of the widget.

Embodiments described herein are generally directed multi-lumen catheters that include one or more filler in a portion thereof. In some aspects, the one or more filler may act as a plug to block flow through the lumen in which it is located. In some aspects, the filler may prevent the creation or growth of bacterial or thrombogenicity in the space between a lumen exit and a tip of the catheter. In some aspects, the one or more filler may be a monofilament formed by one or more extrusion process.

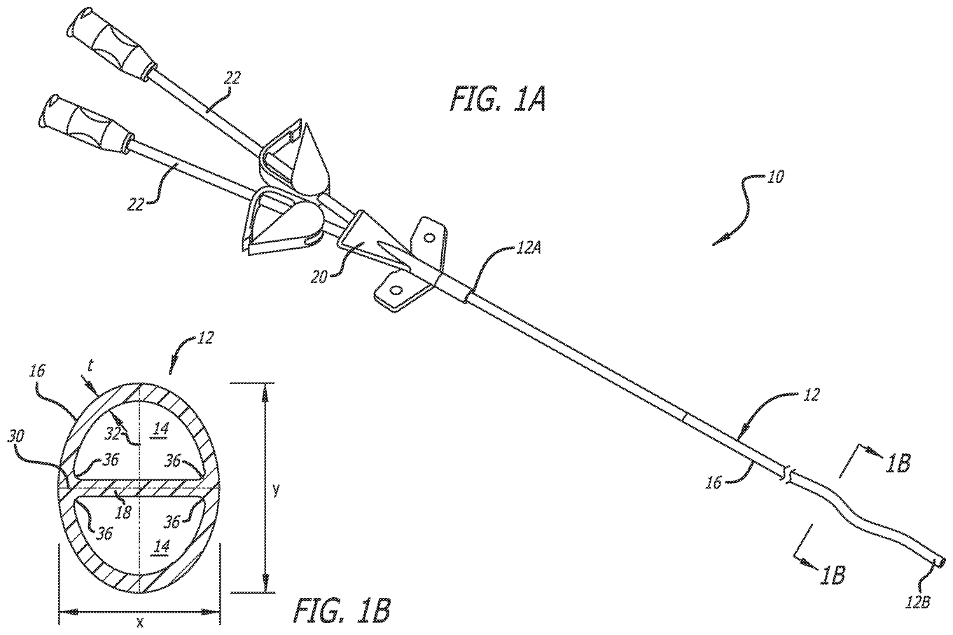

Reference is first made to FIG. 1A, which depicts a catheter assembly, generally designated at 10, configured in accordance with one embodiment. As shown, the catheter assembly (“catheter”) 10 includes an elongate catheter tube 12 formed by an outer wall 16 which, together with a septum 18 (FIG. 1B) defines two (or more) lumens 14 longitudinally extending between a proximal end 12A and a distal end 12B of the tube. A bifurcation 20 mates with the catheter tube 12 at the proximal end 12A thereof to provide fluid communication between the catheter tube and one or more extension legs 22.

FIG. 1B is a cross-sectional view of the catheter tube 12 of FIG. 1A, according to the present embodiment, wherein the catheter tube is aligned such that the width thereof extends along an x-axis and the height thereof extends along a y-axis, the x and y-axes being depicted in FIG. 1B and selected succeeding figures. As shown, the tube 12 cross-sectionally defines two lumens in a generally double-D configuration. Note that the corners 36 of each lumen 14 where the septum 18 joins with the outer wall 16 are rounded to provide more laminar flow through the lumen. The tube 12 further cross-sectionally defines an elliptical profile, further defined by a minor axis 30 parallel to the x-axis and a major axis 32 parallel to the y-axis, in the orientation shown in FIG. 1B. Both the minor and major axes 30, 32 are measured from the perimeter, or outer diameter (“OD) of the catheter tube outer wall 16 in the present embodiment. Note that, though in the present embodiment the elliptical nature of the catheter tube profile extends substantially the entire length of the tube, in other embodiments the elliptical profile can be included on less than the entire catheter tube length.

Because of the elliptical nature of the catheter tube 12 as shown here, the width of the septum 18 in extending between opposite sides of the outer wall 16 to help define the two lumens 14 is shorter relative to the septum width in a correspondingly sized catheter tube with a circular cross-sectional profile. This in turn enables the septum to be stiffer in the elliptical catheter tube, which in turn helps prevent undesired septum deflection when pressure differentials exist between the lumens, such as in dialysis applications for instance. Optionally, this also enables the septum to be made thinner without compromising the rate of septum deflection over a septum of a round catheter tube.

Note that in the present embodiment shown in FIG. 1A, the bifurcation 20 provides fluid paths to establish fluid communication between the cross-sectionally round extension legs 22 and the lumens of the elliptically shaped catheter tube 12. As such, the fluid paths defined in the bifurcation 20 in one embodiment can transition in cross-sectional shape from substantially round proximate the extension legs 22 to substantially elliptical proximate the bifurcation connection point with the proximal end 12A of the catheter tube 12. This can in turn further enhance fluid flow for the catheter assembly. In one embodiment, elliptical core pins are employed during manufacture of the bifurcation and catheter tube to provide properly shaped fluid paths within the bifurcation. The bifurcation in other embodiments can define other shaped fluid paths. Indeed, in one embodiment both the catheter tube and the extension legs can include elliptical cross-sectional shapes, and as such the bifurcation can define substantially elliptical cross-sectional fluid paths. These and other modifications are contemplated.

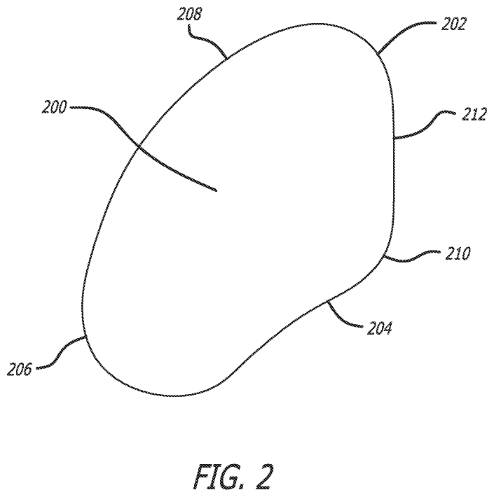

FIG. 2 illustrates a cross-sectional view of a filler 200 for use in plugging, blocking, or partially occluding a portion of a lumen 14 in catheter tube 12 in accordance with an embodiment of the disclosure. Filler 200 may be formed as a monofilament for use in plugging a portion of a catheter lumen during manufacture or use of catheter tube 12. In one aspect, the filler 200 may be formed by an extrusion process such as hot extrusion, cold extrusion, warm extrusion, friction extrusion, micro extrusion, direct extrusion, indirect extrusion, hydrostatic extrusion, impact extrusion, and other extrusion processes that allow for the formation of fillers having a desired cross-section. In another aspect, the filler 200 may be formed by machining or molding suitable materials.

Filler 200 may include any biocompatible polymeric material suitable for occluding or partially occluding a catheter lumen. Such materials may include, but are not limited to, silicone, nylon, polyurethane, polyethylene terephthalate, latex, plastics, thermosets, and thermoplastic elastomers. In some aspects, filler 200 may be formed of a suitable biocompatible metal. In some aspects, filler 200 may include a radiopaque material.

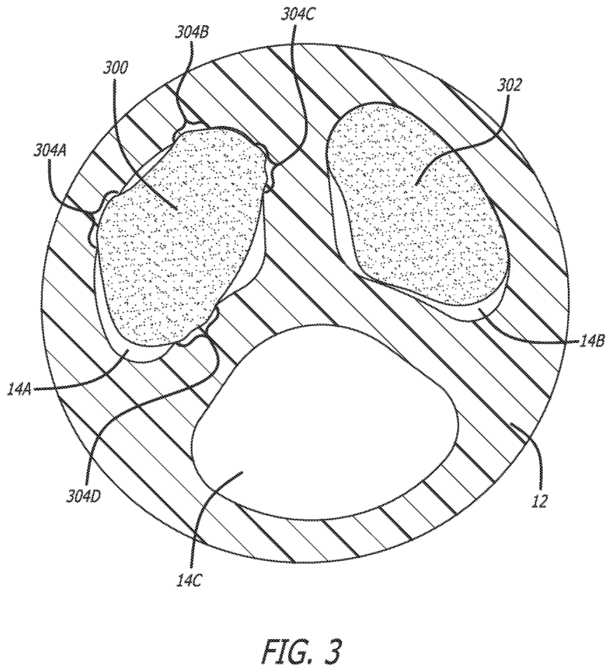

The filler 200 may include a kidney-shaped cross-section. The filler 200 may include a first convex portion 202, a second convex portion 206, a third convex portion 208, and a fourth convex portion 210. The filler 200 may also include a first concave portion 204 and generally straight portion 212. In some aspects, each of the first convex portion 202, second convex portion 206, third convex portion 208, fourth convex portion 210, and first concave portion 204 may each be defined by curve having a radius. In some aspects, at least some of the first convex portion 202, second convex portion 206, third convex portion 208, fourth convex portion 210, and first concave portion 204 may have a curve sharing the same or similar radius. In other aspects, each of the first convex portion 202, second convex portion 206, third convex portion 208, fourth convex portion 210, and first concave portion 204 may have curves including different radiuses. The unique cross-sectional shape of the filler 200, which may be referred to as a “kidney-shaped cross-section,” is specifically configured to replicate the shape of a lumen into which the filler 200 may be placed. The intent of the kidney-shaped cross-section was to fill most, if not all, of the lumen. An example of this is seen in FIG. 3. The filler 200 may have a length that extends the length of or substantially the length of a catheter lumen. Alternatively, the filler 200 may have varying lengths such as 1 inch, 3 inches, 6 inches, 12 inches, etc.

FIG. 3 is a cross-sectional view of a catheter tube 12 including the fillers 300, 302 in accordance with an embodiment. Catheter tube 12 may include a plurality of lumens including a proximal lumen 14A, a medial lumen 14B, and a distal lumen 14C. The proximal lumen 14A may extend from a proximal portion of the catheter tube 12 to a most proximal opening situated along a portion of catheter tube 12. The distal lumen 14C may extend from a proximal portion of the catheter tube 12 to a distal most opening situated along a distal portion of catheter tube 12. The medial lumen 14B may extend from a proximal portion of the catheter tube 12 to a medial opening positioned between the most proximal opening and the distal most opening. Each of the proximal lumen 14A and the medial lumen 14B may include a kidney-shaped cross-section. The distal lumen 14C as shown includes a generally triangular-shaped cross-section having rounded corners. In some aspects, the distal lumen 14C may include a kidney-shaped cross-section that is substantially similar to the cross-section of proximal lumen 14A and medial lumen 14B or the proximal lumen 14A and the medial lumen 14B may include a generally triangular-shaped cross-section.

In order to plug or otherwise fill a portion of the proximal lumen 14A and the medial lumen 14B of the catheter tube 12, fillers 300, 302 may be inserted therein, where the fillers 300, 302 may correspond to the filler 200 discussed with respect to FIG. 2. Fillers 300, 302 may include a kidney-shape cross-section and be substantially similar to filler 200 illustrated in FIG. 2. In fact, the cross-sectional shape of the fillers 300, 302 is intended to block most, if not all, of the lumens 14A, 14B. The cross-section of filers 300, 302 may correspond to a cross-section of proximal lumen 14A and medial lumen 14B. In some aspects, the portion of proximal lumen 14A or medial lumen 14B containing fillers 300, 302 mat be located in a distal section of the catheter tube 12. In some aspects, the portion of proximal lumen 14A or medial lumen 14B containing fillers 300, 302 may be located in a medial section of the catheter tube 12. In some aspects, the portion of proximal lumen 14A or medial lumen 14B containing fillers 300, 302 may be located in a proximal section of the catheter tube 12.

When properly positioned, fillers 300, 302 that include the same or similar cross-section of proximal lumen 14A and medial lumen 14B may effectively plug, block, or occlude the lumens 14A, 14B. As seen in FIG. 3, the filler 204 is positioned properly within the lumen 14B. However, as is often the case, either or both of the fillers 300, 302 may be improperly positioned during insertion into the lumens 14A, 14B. As seen in FIG. 3, the filler 300 is improperly positioned, e.g., positioned in a mirrored or reversed positioning. The improper positioning of the filler 300 reduces the effectiveness of plugging the lumen 14A and also causes bulging of a wall of the catheter tube 12 adjacent the misaligned filler. For example, the improperly positioned filler 300 causes bulging at points 304A, 304B, 304C, and 304D. This may negatively impact the structural integrity of the catheter tube 12 and specifically the walls of the lumens included therein.

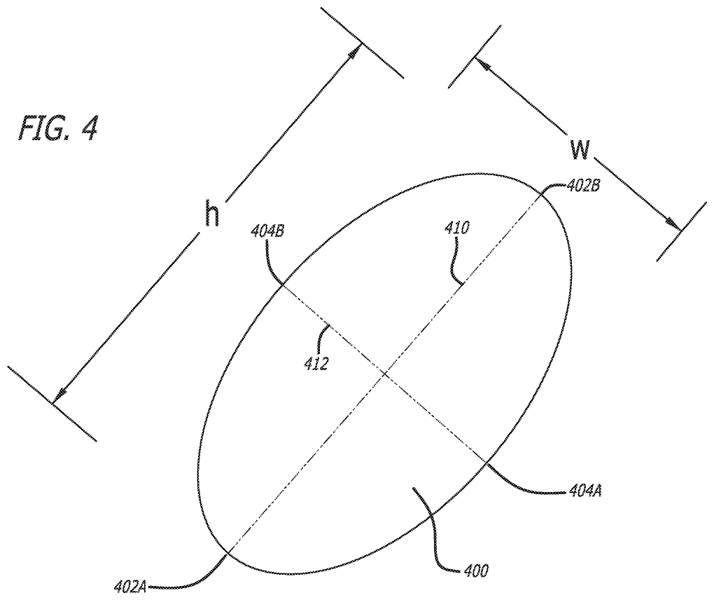

FIG. 4 is a cross-sectional view of a filler 400 in accordance with an embodiment of the disclosure. Filler 400 may be formed as a monofilament for use in plugging a portion of lumen during manufacture or use of catheter tube 12. In one aspect, the filler 400 may be formed by an extrusion process such as hot extrusion, cold extrusion, warm extrusion, friction extrusion, micro extrusion, direct extrusion, indirect extrusion, hydrostatic extrusion, impact extrusion, and other extrusion processes that allow for the formation of fillers having a desired cross-section. In another aspect, the filler 400 may be formed by machining or molding suitable materials.

Filler 400 may include any biocompatible polymeric or other material suitable for occluding or partially occluding a catheter lumen. Such materials may include, but are not limited to, silicone, nylon, polyurethane, polyethylene terephthalate, latex, plastics, and thermoplastic elastomers. In some aspects, filler 400 may be formed of a suitable biocompatible metal. In some aspects, filler 400 may include a radiopaque material.

Unlike filler 200, filler 400 does not have a cross-section similar to a lumen into which it is inserted. Rather, filler 400 includes a symmetrical cross-section. As illustrated in FIG. 4, filler 400 includes an oval cross-section having a vertices 402A-402B, a co-vertices 404A-404B. Filler 400 includes a first line of symmetry 410 corresponding to an axis along a height h of the filler 400 (e.g., measured along a major axis 410). A second line of symmetry 412 corresponds to an axis along a width w of the filler 400 (e.g., measured along a minor axis 412). In some embodiments, the first line of symmetry 410 has a length within a range of 0.035-0.050 inches. In some embodiments, the first line of symmetry 410 has a range of 0.041±0.002 inches. In one particular embodiment, the first line of symmetry 410 has a length of 0.041 inches. In some embodiments, the second line of symmetry 412 has a length within a range of 0.0175-0.035 inches. In some embodiments, the second line symmetry 412 has a range of 0.0250±0.0015 inches. In one particular embodiment, the second line of symmetry 412 has a length of 0.0250 inches.

While FIG. 4 illustrates the filler 400 having an oval cross-section, it is understood that the filler can include other shapes having at least one line of symmetry. For example, filler 400 may have a cross-sectional shape of an oval, a rectangle, a square, a triangle, a circle, or a polygon.

The symmetric configuration of filler 400 ensures that the filler 400 is not loaded into a lumen in an improper orientation, for example, in contrast to the misalignment of filler 300 within the lumen 14A as seen FIG. 3. As noted above, misalignment of the filler 300 disadvantageously causes bulging of lumen walls. Additionally, the symmetrical shape of filler 400 allows for simplification of an extrusion die and increases ease of manufacturing.

FIG. 5 is a cross-sectional view of a catheter tube including a plurality of fillers 500, 502 in accordance with an embodiment. Fillers 500, 502 may be substantially similar to filler 400 illustrated in FIG. 4. Catheter tube 12 may include a plurality of lumens including a proximal lumen 14A, a medial lumen 14B, and a distal lumen 14C. The proximal lumen 14A may extend from a proximal portion of the catheter tube 12 to a most proximal opening situated along a portion of catheter tube 12. The distal lumen 14C may extend from a proximal portion of the catheter tube 12 to a distal most opening situated along a distal portion of catheter tube 12. The medial lumen 14B may extend from a proximal portion of the catheter tube 12 to a medial opening positioned between the most proximal opening and the distal most opening. Each of the proximal lumen 14A and the medial lumen 14B may include a kidney-shaped cross-section. The distal lumen 14C as shown includes a generally triangular shaped cross-section having rounded corners. In some aspects, the distal lumen 14C may include a kidney-shaped cross-section similar to the cross-section of proximal lumen 14A and medical lumen 14B.

Filler 402 may be inserted in a portion of proximal lumen 14A in order to plug or block the lumen 14A at a desired location along the length thereof. In some aspects, proximal lumen 14A may be blocked in a distal portion, medial portion, or proximal portion of catheter tube 12. The symmetrical shape of filler 402 allows for placement into the proximal lumen 14A without worry of incorrect insertion. Similarly, filler 404 may be inserted in a portion of medial lumen 14B in order to plug or block the lumen 14B at a desired location along the length thereof. In some aspects, medial lumen 14B may be blocked in a distal portion, medial portion, or proximal portion of catheter tube 12. The symmetrical shape of filler 404 allows for placement into the proximal lumen 14B with little to no likelihood of improperly positioning the fillers 500, 502 within the lumens 14A, 14B. Fillers 500, 502 may have the same length or different lengths and may extend along a desired length of lumens 14A, 14B.

In some aspects, the portions of proximal lumen 14A and medial lumen 14B containing fillers 402 and 404, respectively, may be at the same location along a length of the catheter tube 12. In some aspects, the portions of proximal lumen 14A and medial lumen 14B containing fillers 402 and 404, respectively, may be at different locations along a length of the catheter tube 12. For example, filler 402 may be in a distal section of the catheter tube 12 while filler 404 may be positioned in a medial section of the catheter tube 12. In some aspects, proximal lumen 14A or medial lumen 14B may contain more than one filler to plug or block more than one section of a lumen.

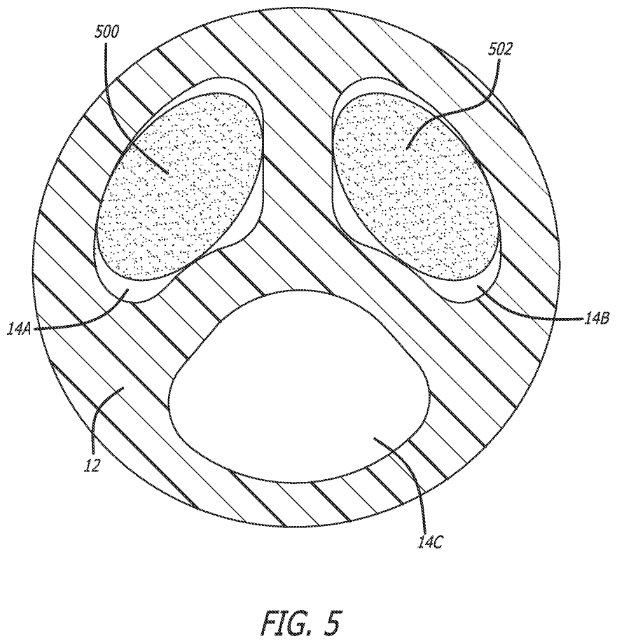

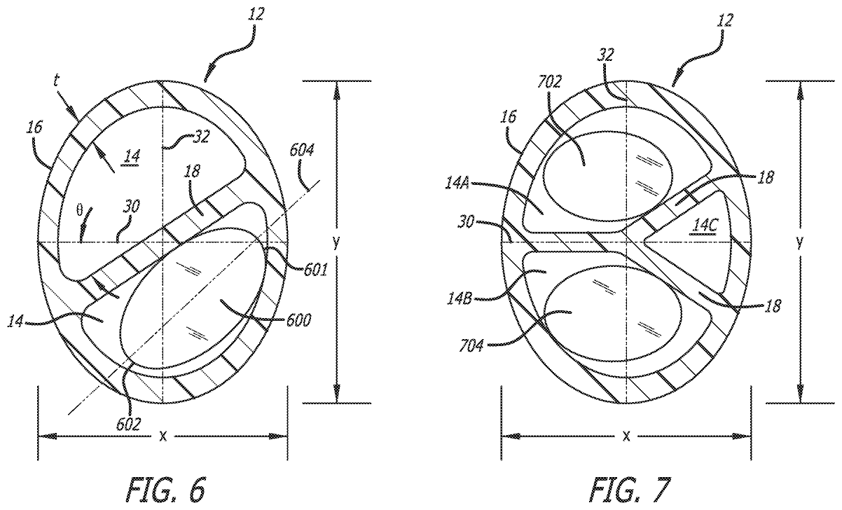

FIG. 6 shows a cross-section of a catheter tube 12 according to another embodiment, wherein the elliptical profile is retained as defined by the minor and major axes 30, 32, but the septum 18 of the tube is slanted so as to define an angle θ with the minor axis 30. The slanted septum configuration illustrated in FIG. 6 provides in one embodiment relatively greater stability for the distal tip of the catheter during fluid infusion therethrough. The slanted septum configuration further balances the principal axis of I for the catheter tube, thus reducing the likelihood of the catheter tube to roll or bend in only one direction.

The catheter tube 12 of FIG. 6 includes a filler 600 positioned in a portion of lumen 14. Filler 600 may have an oval cross-section that includes a first line of symmetry 604. A first end 601 includes a radius that is smaller than a radius of a second end 602. As discussed above in reference to fillers 200, 400, filler 600 may be formed as a monofilament of a desired biocompatible material. Filler 600 may extend along a desired length of lumen 14.

FIG. 7 shows a cross-section of a catheter tube 12 according to another embodiment, wherein the elliptical profile is retained as defined by the minor and major axes 30, 32, but the tube defines three lumens 14A, 14B, and 14C in a triple lumen configuration. As shown, the septum 18 splits to border either side of the generally triangular third lumen 14C. As was the case with dual lumen catheter tube, the triple lumen configuration shown here improves flow rates for each of the lumens 14A, 14B, and 14C due to the elliptical catheter tube profile. It is noted that in one embodiment, one or more of the lumens 14A-14C can be configured for relatively high fluid flow rates therethrough, commonly referred to as power injection. Indeed, in the other embodiments herein described, one or more of the lumens of the catheter tube can be configured to withstand power injection.

Lumens 14A, 14B are illustrated as containing fillers 702 and 704 respectively along a portion thereof. Fillers 702 and 704 may be similar to filler 400 discussed above. However, fillers 702 and 704 may include similar cross-sections but have different sizes. For example, fillers 702, 704 may have an oval cross-section, but filler 702 may have a width or length that is smaller than filler 704. Such variations allow for different levels of blockage or plugging for each lumen 14A, 14B. Fillers 702, 704 may have the same length or different lengths and may extend along a desired length of lumens 14A, 14B.

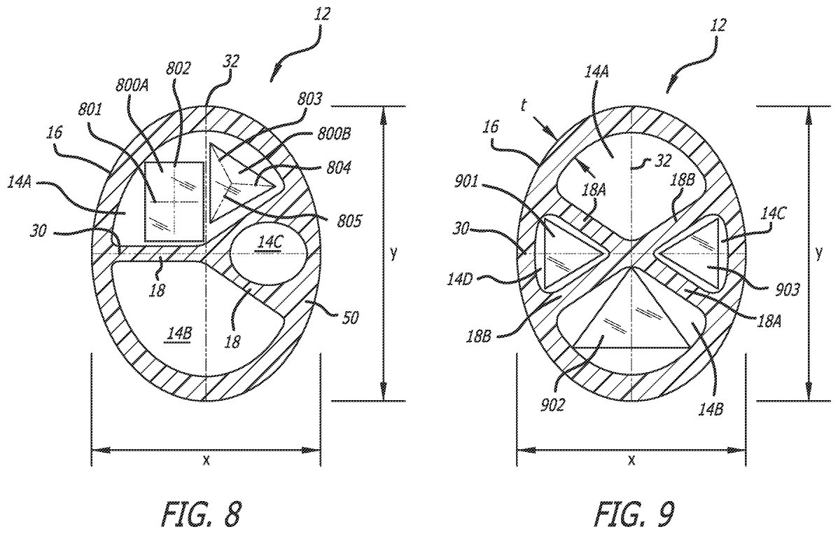

FIG. 8 shows a cross-section of a catheter tube 12 according to another embodiment, wherein the elliptical profile is retained as defined by the minor and major axes 30, 32, and the tube defines three lumens 14A, 14B, and 14C in a triple lumen configuration, as in FIG. 7, wherein the septum 18 splits to border either side of the now circular third lumen 14C. Again, and as was the case with dual lumen catheter tube, the triple lumen configuration shown here improves flow rates for each of the lumens 14A, 14B, and 14C due to the elliptical catheter tube profile.

In contrast to the configuration of FIG. 7, the catheter tube 12 in FIG. 8 includes a portion 50 defining the portions of the outer wall 16 and the septum 18 that bound the third lumen 14C. The portion 50 extends longitudinally the length of the catheter tube and includes a relatively harder material than that of the material defining the rest of the septum 18 and outer 16. This relatively harder material reinforces the third lumen 14C to enable it to withstand the high fluid pressures typically associated with power injection.

Also, in one embodiment the material included in the portion 50 enables the portions of the outer wall 16 and septum 18 thinner than what would otherwise be possible, in turn enabling the other lumens 14A and 14B to be larger than they would otherwise be. In other embodiments, the material defining the portion 50 can also be stiffer and/or include greater tensile strength relative to the other portions of the outer wall and septum so as to provide the desired characteristics for the third lumen. In yet another embodiment, the portion 50 can extend to encompass the entirety of the septum 18.

Note that, as was the case with the elliptical dual lumen configurations above, the width of the septa 18 of triple and quad lumen configurations discussed here are shorter relative to the septa in correspondingly sized catheter tubes with a circular cross-sectional profiles. Again, this stiffens the septum, which in turn helps prevent undesired septum deflection when pressure differentials exist between the lumens.

In one embodiment, for example, the catheter portion 50 includes a material of hardness of about 100 Shore A, while the remaining portions of the catheter tube 12 include a material of hardness of about 85 Shore A. Thermoplastic polyurethanes including those sold under the names TECOTHANE® and CARBOTHANE® are non-limiting examples of materials that can be configured to meet the above or other desired hardness characteristics for the portion 50 and remaining portions of the catheter tube 12. The catheter tube 12 as illustrated and discussed herein can be formed via co-extrusion, insert extrusion, and other suitable methods.

Additionally, the catheter tube 12 in FIG. 8 may include a plurality of fillers 800A, 800B positioned in the same lumen 14A along a portion thereof. Filler 800A may include a square or rectangular cross-section having a first line of symmetry 801 and a second line of symmetry 802. Filler 800B may include a triangular cross-section having a first line of symmetry 803, a second line of symmetry 804, and a third line of symmetry 805. Fillers 80A, 800B may be inserted into lumen 14A simultaneously at the same portion of lumen 14A to block or plug lumen 14A together. Using a plurality of fillers having various cross-sections within the same lumen, each filler having at least one line symmetry, may allow for more compete blockage of a lumen at a desired location.

FIG. 9 shows a cross-section of a catheter tube 12 according to another embodiment, wherein the elliptical profile is retained as defined by the minor and major axes 30, 32, but the tube now defines four lumens 14A, 14B, 14C, and 14D in a quad lumen configuration. As shown, two septa 18A and 18B intersect one another to define, together with the outer wall 16, the four lumens 14A-14D. As before one, two, or more of the lumens 14A-14D can be configured for power injection and flow therethrough is optimized due to the elliptical aspect ratio of the catheter tube 12.

As illustrated in FIG. 9, catheter tube 12 includes a plurality of fillers 901, 902, 903. The fillers 901, 902, 903 each include a triangular cross-section and include at least one line of symmetry. For example, fillers 901 and 903 share a line of symmetry along minor axis 30, and filler 902 includes a line of symmetry along major axis 32. Fillers 901 and 903 may have a first size, and filler 902 may have a second size larger than the first size. Fillers 901, 902, 903 may be located at different locations or the same location along the length of catheter tube 12. In one aspect, the lengths of fillers 901, 902, 903 may be the same. In another aspect, the lengths of fillers 901, 902, 903 may be different.

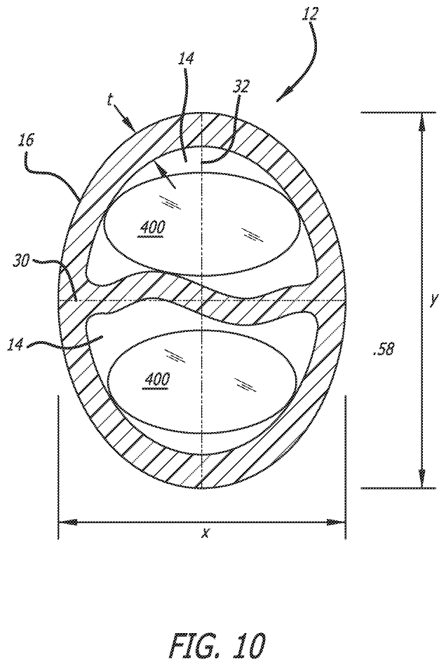

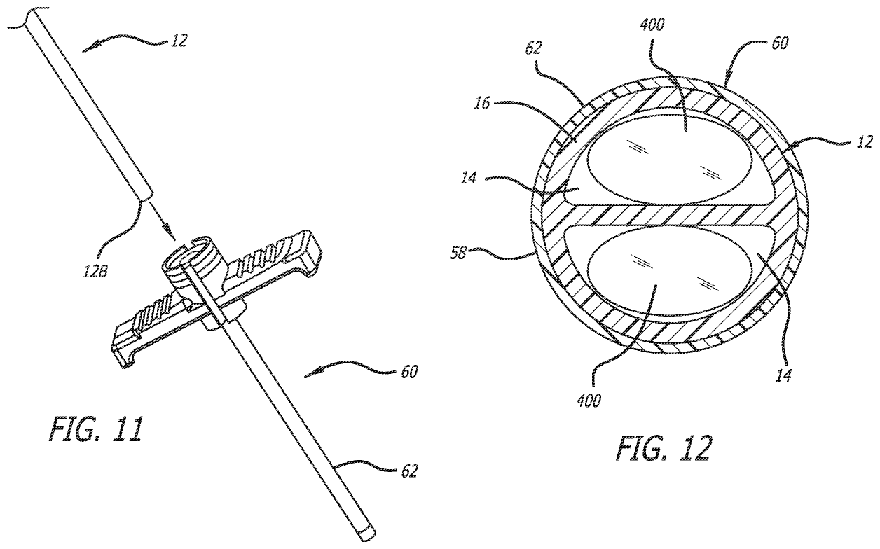

FIG. 10 shows a cross-section of a catheter tube 12 according to another embodiment, wherein the elliptical profile is retained as defined by the minor and major axes 30, 32. A septum 58 dividing the two lumens 14 is also shown. Each of the lumens 14 may include a filler 400 positioned along a portion thereof. The septum 58 is initially slackened when the catheter tube 12 is in a rest state as shown in FIG. 10. This enables the elliptical catheter tube 12 to be fed through a round catheter introducer, such as the introducer 60 shown in FIG. 11. In particular, FIG. 11 shows that catheter tube 12 of FIG. 10 being introduced into the proximal end of the introducer 60. The introducer 60 includes a round body 62, a portion of which is initially disposed within a vessel of the patient.

Introduction of the elliptical catheter tube 12 into the round introducer body 62 forces the tube outer wall 16 to deform into the round shape of the introducer body and conform to a surface of fillers 400, which leads to blocking, plugging, or occlusion of the lumens 14. Because of the initially slackened state of the septum 58, the catheter tube 12 is able to be deformed from the elliptical to the circular shape when it passes into the round introducer body 62. This causes the initially slackened septum 58 to be stretched taut as the outer body 16 of the catheter tube 12 is forced into the circular shape, as shown in FIG. 12, which shows the catheter tube 12 disposed within the introducer body 62. This enables the catheter tube 12 to be inserted into the patient's vessel, after which the introducer 60 can be removed from the vessel and the catheter tube resiliently returns to its elliptical aspect profile (FIG. 10).

In one embodiment, a proximal portion of the introducer and/or introducer body can include a transition region that gradually changes from an elliptical profile to a round profile so as to ease insertion of the initially elliptical catheter tube into the introducer. In another embodiment, an elliptical introducer may be used to place the elliptical catheter tube into the patient's vasculature. Note that the slackened shape of the septum can vary from the wavy configuration shown in FIG. 10, including a bowed or arced shaped, for instance.

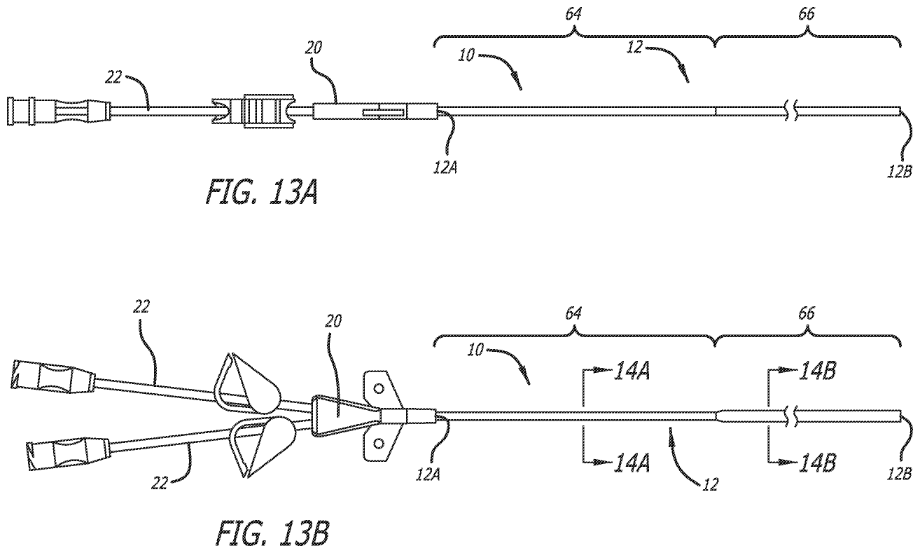

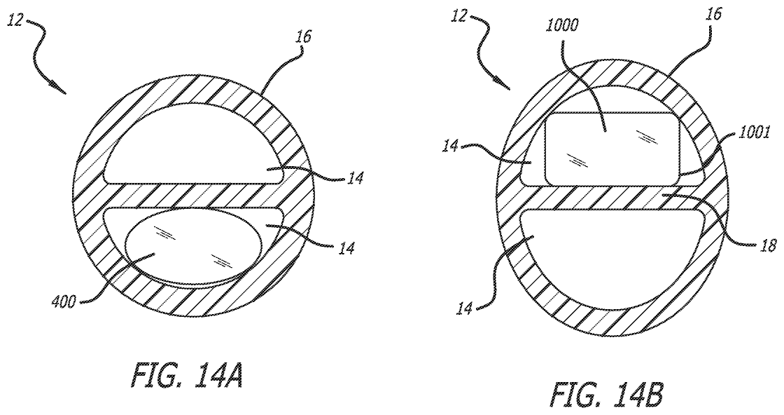

FIGS. 13A and 13B show side and top views, respectively, of a catheter assembly 10 according to one embodiment, wherein the dual lumen catheter tube 12 includes a proximal portion 64 extending distally from the bifurcation 20 and a distal portion 66 extending distally from the distal termination of the proximal portion to the distal tip of the catheter tube. In particular, the proximal portion 64 of the illustrated embodiment includes a circular cross-sectional profile, as seen by the sectional view of FIG. 14A. The distal portion 66 of the catheter tube includes an elliptical cross-sectional profile, similar to the configuration shown in FIG. 1B, as seen by the sectional view of FIG. 14B.

Catheter tube 12 may also include one or more fillers 400, 1000 located at various portions of lumens 14. As illustrated in FIG. 14A, filler 400 may be positioned in a portion of lumen 14 at a proximal portion 64 of catheter tube. Additionally, in distal portion 66, filler 1000 may be located in lumen 14′. Filler 1000 may include a rectangular cross-section having rounded corners. In some aspects, filler 400 and filler 1000 may be positioned at different locations along the same lumen.

Observation of FIGS. 13A and 13B shows that the distal portion 66 increases in diameter with respect to the proximal portion 64, best seen in the top view of FIG. 13B, owing to the elliptical nature of the distal portion. In one embodiment, this provides desirably low hydraulic resistance in the distal portion of each lumen 14, as well as enhanced power injection behavior, e.g., relatively low power injection pressures and relatively greater distal tip stability. Moreover, the round proximal portion 64 of the catheter tube of FIGS. 13A and 13B is less likely to flip when the catheter tube is maneuvered within the vasculature during and after placement procedures.

As such, it is appreciated that a portion of the catheter tube may include an elliptical profile while other portions do not. In another embodiment it is appreciated that the positions of the circular and elliptical portions of the catheter tube can be reversed. In yet another embodiment, the average diameter of the proximal or distal portion of the catheter tube can increase relative the other. More generally, the size, number, length, lumen number, and placement of one or more elliptical portions of the catheter tube can vary as appreciated by one skilled in the art. Moreover, it is understood that the nature and/or degree/magnitude of the elliptical profile can vary over the length of the catheter tube. Further details regarding catheters that include features for enhancing the stability of a distal tip thereof can be found in U.S. application Ser. No. 13/209,270, filed Aug. 12, 2011, and entitled “Trimmable Catheter Including Distal Portion Stability Features,” which is incorporated herein by reference in its entirety.

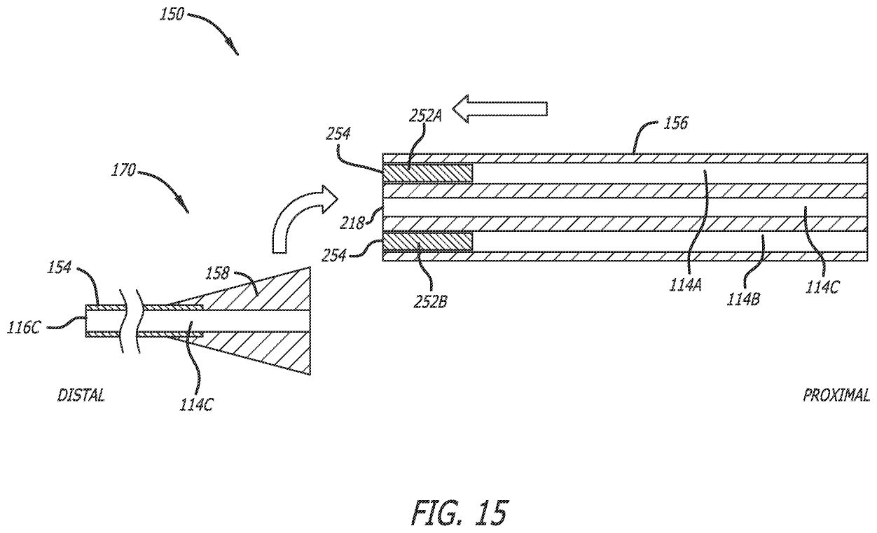

FIG. 15 shows an exemplary method of manufacturing a catheter 150 including a distal tip structure 170 having one or both of the PIV section 154 and the dilator section 158. In an exemplary method of coupling a distal tip structure 170 to a central venous catheter (CVC) section 156 to form a catheter body 152, termed “tipping,” the CVC section 156 can be formed having one or more lumen 114. In an embodiment, the CVC section 156 can be extruded and trimmed to a desired length. It will be appreciated, however, that other methods of forming a CVC section 156 are also contemplated. As shown in FIG. 15, a triple lumen CVC section 156 is provided including a first (proximal) lumen 114A, a second (medial) lumen 114B, and a third (distal) lumen 114C. However, it will be appreciated that other single or multi-lumen catheters 150 are also contemplated. To note, the lumen 114A, 114B, 114C of the CVC section 156 can be arranged radially about a central axis of the CVC section 156. In FIG. 15, the lumens 114A, 114B, 114C are shown adjacent to each other for clarity. It will be appreciated, however, that other configurations of multi-lumen catheters 150 are also contemplated.

In an embodiment, a plug or filler can be disposed into one or both of the proximal lumen 114A and the medial lumen 114B. For example, a first plug or filler 252A can be disposed within a distal end of a first lumen 114A, and a second plug or filler 252B can be disposed within a distal end of a second lumen 114B. First plug or filler 252A or second plug or filler 252B may take the form of any filler discussed above and may include a filler having at least one line of symmetry. A distal tip 254 of the plug 252 can align with a distal end 218 of the CVC section 156. Optionally, a distal tip 254 of the plug 252 can be trimmed to align with a distal end 218 of the CVC section 156.

The distal tip structure 170 can then be coupled with a distal end 218 of the CVC section 156 using adhesive, bonding, solvent bonding, welding or the like. A lumen of the distal tip structure 170 can align with a lumen of the CVC section 156 to form a distal lumen 114C extending to a distal lumen aperture 116C. The first plug or filler 252A can seal the proximal lumen 114A, and the second plug or filler 252B can seal the medial lumen 114B, proximally of the dilator portion 158. The proximal lumen aperture 116A can then be formed through a wall of the CVC section 156 and communicate with the proximal lumen 114A. The medial lumen aperture 116B can then be formed through a wall of the CVC section 156 and communicate with the medial lumen 116B.

It should be understood that these and other variations of the principles described herein are contemplated and that the cross-sectional profiles of the multi-lumen catheter tubes disclosed herein can vary as appreciated by one skilled in the art.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system, comprising:
a catheter comprising:
an elongate tube including a distal end and defining a plurality of lumens, and
a distal tip structure located adjacent the distal end; and
a first plug of filler configured for positioning in a first portion of a first lumen of the plurality of lumens proximal the distal tip, wherein the first plug is defined as having an oval cross-sectional shape.

2. The system of claim 1, further comprising a second plug of filler positioned in a second portion of one of the plurality of lumens, wherein the second plug is defined as having the oval cross-sectional shape.

3. The system of claim 2, wherein the first plug and second plug are positioned in different lumens.

4. The system of claim 1, wherein the first plug is formed of a biocompatible polymeric material.

5. The system of claim 4, wherein the biocompatible polymeric material includes one or more of silicone, nylon, polyurethane, polyethylene terephthalate, a latex, a plastic, a thermoset, or a thermoplastic elastomer.

6. The system of claim 1, wherein the first plug is formed of a radiopaque material.

7. The system of claim 1, wherein the first plug includes a cross-section having a first line of symmetry.

8. The system of claim 7, wherein a length of the first line of symmetry is within a range of 0.035-0.050 inches.

9. The system of claim 8, wherein the length of the first line of symmetry is 0.041+0.002 inches.

10. The system of claim 7, wherein the first plug includes a cross-section having a second line of symmetry.

11. The system of claim 10, wherein a length of the second line of symmetry is within a range of 0.0175-0.035 inches.

12. The system of claim 11, wherein the length of the second line of symmetry is 0.0250+0.0015 inches.

13. The system of claim 1, wherein the oval cross-sectional shape of the first plug includes a first vertex and a second vertex, wherein the first plug is configured such that either the first vertex or the second vertex may be positioned near an upper side of the first lumen without causing bulging of a wall of the lumen.

14. The system of claim 1, wherein the first portion is located of the first lumen in a distal section of the catheter.

15. The system of claim 1, wherein the first portion of the first lumen is located in a medial section of the catheter.

16. The system of claim 1, wherein the first portion of the first lumen is located in a proximal section of the catheter.

17. The system of claim 1, wherein the plurality of lumens includes the first lumen, a second lumen, a third lumen, and a fourth lumen, wherein at least two lumens of the plurality of lumens are bisected by one of a minor axis or a major axis of the catheter, at least one lumen of the plurality of lumens being power injectable.

18. The system of claim 1, wherein a proximal portion of the elongate tube defines a circular cross-section and a distal portion of the elongate tube defines an elliptical cross-section.

19. The system of claim 1, wherein a proximal portion of the elongate tube defines an elliptical cross-section and a distal portion of the elongate tube defines a circular cross-section.

20. The system of claim 1, wherein the plurality of lumens include a cross-sectional profile that is generally triangular or kidney shaped.
